(19) United States
(12) Patent Application Publication
(10) Pub. No.: US 2026/0000284 A1
(43) Pub. Date: Jan. 1, 2026

(54) ENDOSCOPE CAMERA INTERFERENCE MITIGATION

(71) Applicant: Lumenis Ltd., Yokneam Illit (IL)

(72) Inventor: Tal Waisman, Haifa (IL)

(73) Assignee: Lumenis Ltd., Yokneam Illit (IL)

(21) Appl. No.: 18/756,839

(22) Filed: Jun. 27, 2024

(51) Int. Cl. A61B 1/045; A61B 1/00; A61B 17/00; A61B 18/00; A61B 18/26

(52) U.S. Cl. CPC A61B 1/045; A61B 1/00006; A61B 18/26; A61B 2017/0019; A61B 2017/00199; A61B 2018/00982

(57) ABSTRACT

The present disclosure provides an endoscopic laser lithotripsy device and methods for such a device where image acquisition, lasing, illumination, and/or image processing is configured to mitigate distortions in the captured image resulting from the laser.

### CROSS-REFERENCE TO RELATED APPLICATIONS

This application claims the benefit of U.S. Provisional Patent Application Ser. No. 63/510,751 filed on Jun. 28, 2023, the disclosure of which is incorporated herein by reference.

### TECHNICAL FIELD

The present disclosure generally relates to endoscopes and particularly, but not exclusively, to mitigating interference with images capture by a camera of an endoscope when used in conjunction with a surgical laser system.

### BACKGROUND

Endoscopes are used to obtain an internal view of a patient. To that end, endoscopes have one or more cameras. The purpose of the camera is to provide an image of the target (e.g., organ, stone, or the like) on which the medical procedure is being performed or an image in the vicinity of where the medical procedure is to be performed. For example, an endoscope can be used to view the interior of a kidney and/or assess kidney stones in the kidney. As another example, an endoscope can be used to view a ureter possibly having stones, tumors, or strictures. In yet another example, an endoscope can be used to view a bladder and its anatomy such as the ureter openings and possible treatment targets like stones or tumors.

It will be appreciated that interference with image acquisition can cause image quality degradation and/or creation of image artifacts. In severe cases this can cause difficulty in recognizing the required tissue features and characteristics to the level that the safety of continued treatment can be impacted. This may force a physician to determine to stop or pause a treatment procedure until the image (or conditions affecting the image) improves.

Thus, there is a need to improve image capture or rather to mitigate interference with camera operation and/or image capture by the camera.

### BRIEF SUMMARY

This Summary is provided to introduce a selection of concepts in a simplified form that are further described below in the Detailed Description. This Summary is not intended to necessarily identify key features or essential features of the claimed subject matter, nor is it intended as an aid in determining the scope of the claimed subject matter.

The present disclosure provides an endoscopic laser lithotripsy device and methods for such a device where image acquisition and/or processing is configured to mitigate interference from the laser.

Some embodiments of the disclosure can be implemented as a method for an endoscope camera controller. The method can comprise receiving, at a processor of an endoscope console, an indication of a pulse start control signal and one or more laser pulse characteristics, the pulse start control signal to define an initiation of lasing by the lasing console and the one or more laser pulse characteristics to define the lasing; determining, by the processor, an integration timing period of a camera sensor of an endoscope based on the pulse start control signal and the one or more laser pulse characteristics; and generating, by the processor, an integration control signal, the integration control signal to cause the camera sensor to accumulate charge during integration timing period.

In further embodiments, the method can comprise sending the integration control signal to shutter gating circuitry of the endoscope, the shutter gating circuitry to control a shutter of the endoscope camera.

In further embodiments of the method, the one or more laser pulse characteristics define a frequency of pulses to be generated by the lasing console.

In further embodiments of the method, the one or more laser pulse characteristics define a pulse width of the pulses.

In further embodiments of the method, determining the integration timing period of the camera sensor comprises determining a maximum duration of integration of the camera sensor based on a frame rate of the camera; and determining the integration timing period as the difference of the maximum duration of integration of the camera and the pulse width.

In further embodiments of the method, the camera is a global shutter style camera or a rolling shutter style camera.

In further embodiments of the method, the lasing console comprises a Thulium fiber laser source or a Holmium laser source.

Some embodiments of the disclosure can be implemented as a method for a laser console. The method can comprise receiving, at a processor of a laser console, an indication of settings of an endoscope camera; determining, by the processor, laser pulse characteristics and a pulse start time for a laser source of the laser console based on the settings; and causing the laser source to generate pulses of light based on the laser pulse characteristics and the pulse start time.

In further embodiments of the method, the settings comprise an indication of a frame rate and a vertical blanking period of the endoscope camera.

In further embodiments of the method, determining the pulse start time is based on the vertical blanking period.

In further embodiments of the method, determining the laser pulse characteristics comprises determining a frequency of the laser source based on a factor of the frame rate.

Some embodiments of the disclosure can be implemented as a method for an endoscope console. The method can comprise receiving, at a processor of an endoscope console, an indication of settings of an endoscope camera; receiving, at a processor of an endoscope console, an indication of a pulse start time and one or more laser pulse characteristics, the pulse start control signal to define an initiation of lasing by the lasing console and the one or more laser pulse characteristics to define the lasing; receiving, by the processor, a plurality of image frames from a camera of an endoscope coupled to the endoscope console; identifying, by the processor, at least a portion of a one of the plurality of image frames where lasing by the lasing console overlaps with integration by the camera based on the one or more laser pulse characteristics, the pulse start time, and the settings; identifying, by the processor, at least a portion of another one of the plurality of image frames where lasing by the lasing console does not overlap with integration by the camera based on the one or more laser pulse characteristics, the pulse start time, and the settings; and replacing at least the portion of the one of the plurality of image frames with at least the portion of the other one of the plurality of image frames, wherein the other one of the plurality of image frames is prior in time to the one of the plurality of image frames.

In further embodiments of the method, the settings comprise an indication of a frame rate and a vertical blanking period of the endoscope camera.

In further embodiments of the method, the one or more laser pulse characteristics define a frequency of pulses to be generated by the lasing console.

In further embodiments of the method, the one or more laser pulse characteristics define a pulse width of the pulses.

Some embodiments of the disclosure can be implemented as an endoscope camera controller. The controller can comprise a processor and memory coupled to the processor, the memory comprising instructions, which when executed by the processor cause the endoscope camera controller to receive an indication of a pulse start control signal and one or more laser pulse characteristics, the pulse start control signal to define an initiation of lasing by a lasing console and the one or more laser pulse characteristics to define the lasing; determine an integration timing period of a camera sensor of an endoscope based on the pulse start control signal and the one or more laser pulse characteristics; and generate an integration control signal, the integration control signal to cause the camera sensor to accumulate charge during integration timing period.

In further embodiments of the controller, the instructions when executed by the processor further cause the endoscope camera controller to send the integration control signal to shutter gating circuitry of the endoscope, the shutter gating circuitry to control a shutter of the endoscope camera.

In further embodiments of the controller, the one or more laser pulse characteristics define a frequency of pulses to be generated by the lasing console.

In further embodiments of the controller, the one or more laser pulse characteristics define a pulse width of the pulses.

In further embodiments of the controller, the instructions when executed by the processor further cause the endoscope camera controller to determine a maximum duration of integration of the camera sensor based on a frame rate of the camera; and determine the integration timing period as the difference of the maximum duration of integration of the camera and the pulse width.

In further embodiments of the controller, the camera is a global shutter style camera or a rolling shutter style camera.

In further embodiments of the controller, the lasing console comprises a Thulium fiber laser source or a Holmium laser source.

Some embodiments of the disclosure can be implemented as a laser console. The laser console can comprise a processor and memory coupled to the processor, the memory comprising instructions, which when executed by the processor cause the laser console to receive an indication of settings of an endoscope camera; determine laser pulse characteristics and a pulse start time for a laser source of the laser console based on the settings; and cause the laser source to generate pulses of light based on the laser pulse characteristics and the pulse start time.

In further embodiments of the console, the settings comprise an indication of a frame rate and a vertical blanking period of the endoscope camera.

In further embodiments of the console, the instructions when executed by the processor further cause the laser console to determine the pulse start time based on the vertical blanking period.

In further embodiments of the console, the instructions when executed by the processor further cause the laser console to determine the laser pulse characteristics comprises determining a frequency of the laser source based on a factor of the frame rate.

In further embodiments of the console, the laser source is a Thulium fiber laser source.

In further embodiments of the console, the laser source is a Holmium laser source.

In further embodiments of the console, the endoscope camera is a global shutter style camera or a rolling shutter style camera.

Some embodiments of the disclosure can be implemented as an endoscope console. The console can comprise a processor and memory coupled to the processor, the memory comprising instructions, which when executed by the processor cause the endoscope console to receive an indication of settings of an endoscope camera; receive an indication of a pulse start time and one or more laser pulse characteristics, the pulse start control signal to define an initiation of lasing by the lasing console and the one or more laser pulse characteristics to define the lasing; receive a plurality of image frames from a camera of an endoscope coupled to the endoscope console; identify at least a portion of a one of the plurality of image frames where lasing by the lasing console overlaps with integration by the camera based on the one or more laser pulse characteristics, the pulse start time, and the settings; identify at least a portion of another one of the plurality of image frames where lasing by the lasing console does not overlap with integration by the camera based on the one or more laser pulse characteristics, the pulse start time, and the settings; and replace at least the portion of the one of the plurality of image frames with at least the portion of the other one of the plurality of image frames, wherein the other one of the plurality of image frames is prior in time to the one of the plurality of image frames.

In further embodiments of the console, the settings comprise an indication of a frame rate and a vertical blanking period of the endoscope camera.

In further embodiments of the console, the one or more laser pulse characteristics define a frequency of pulses to be generated by the lasing console.

In further embodiments of the console, the one or more laser pulse characteristics define a pulse width of the pulses.

In further embodiments of the console, the camera is a global shutter style camera or a rolling shutter style camera.

In further embodiments of the console, the lasing console comprises a Thulium fiber laser source or a Holmium laser source.

### BRIEF DESCRIPTION OF THE SEVERAL VIEWS OF THE DRAWINGS

To easily identify the discussion of any element or act, the most significant digit or digits in a reference number refer to the figure number in which that element is first introduced.

FIG. 1A illustrates a lithotripsy system in accordance with at least one embodiment of the present disclosure.

FIG. 1B illustrates a portion of the lithotripsy system of FIG. 1A in greater detail.

FIG. 1C illustrates another portion of the lithotripsy system of FIG. 1A in a particular use case.

FIG. 1D illustrates the other portion of the lithotripsy system of FIG. 1C in another particular use case.

FIGS. 2A and 2B illustrate images of an endoscope showing interference from laser light emitted by a fiber optic cable inserted through a working channel of the endoscope.

FIGS. 3A and 3B illustrate further images of an endoscope showing interference from laser light emitted by a fiber optic cable inserted through a working channel of the endoscope.

FIG. 4 illustrates endoscope system in accordance with at least one embodiment of the present disclosure.

FIG. 5 illustrates a method for synchronizing an integration phase of an endoscope camera with lasing by a laser source in accordance with at least one embodiment of the present disclosure.

FIG. 6 illustrates a method for synchronizing an illumination source of an endoscope camera with lasing by a laser source in accordance with at least one embodiment of the present disclosure.

FIG. 7 illustrates another endoscope system in accordance with at least one embodiment of the present disclosure.

FIG. 8 illustrates a method for synchronizing lasing by a laser source with an integration phase of an endoscope camera in accordance with at least one embodiment of the present disclosure.

FIG. 9 illustrates yet another endoscope system in accordance with at least one embodiment of the present disclosure.

FIG. 10 illustrates a method for generating updated image frames to remove interference from an overlapping of an integration phase of an endoscope camera with lasing of a laser source in accordance with at least one embodiment of the present disclosure.

FIG. 11 illustrates a computer-readable storage medium.

FIG. 12 illustrates a diagrammatic representation of a machine.

### DETAILED DESCRIPTION

The foregoing has broadly outlined the features and technical advantages of the present disclosure such that the following detailed description of the disclosure may be better understood. It is to be appreciated by those skilled in the art that the embodiments disclosed may be readily utilized as a basis for modifying or designing other structures for carrying out the same purposes of the present disclosure. The novel features of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present disclosure.

As noted, the present disclosure provides systems and techniques to mitigate interference of endoscopic camera operation. It will be appreciated that such interference can occur at various points during the timeline of treatment and can disrupt some video frames, while leaving other video frames uninfluenced. In cases of sporadic interference, only temporary effects may be present leaving the viewed scene understandable to the physician. In such a case, this can cause a nuisance to the physician but may still allow for safe operation and not require a pause in the procedure. In other cases, such as where the interference occurs at a higher frequency or where the interference is synchronized with the camera frame rate, the effects on the image may be constant, thereby disrupting all or a portion of the image of the scene. In such a case, the ability to continue safe operation may be unavailable to the physician necessitating a pause or cessation of the procedure.

As an example, medical lasers are used in a variety of endoscopic procedures where laser light is directed to a target through an optical fiber. One such procedure, to address renal calculi (e.g., kidney stones) is ureteral endoscopy, or lithotripsy. In lithotripsy, an endoscopic probe, with a camera or other sensor, is inserted into the patient's urinary tract to locate the calculi for removal. An optical fiber is inserted through a working channel of the probe and laser energy can be directed towards the calculi via the optical fiber to disintegrate the calculi as they are found via the camera.

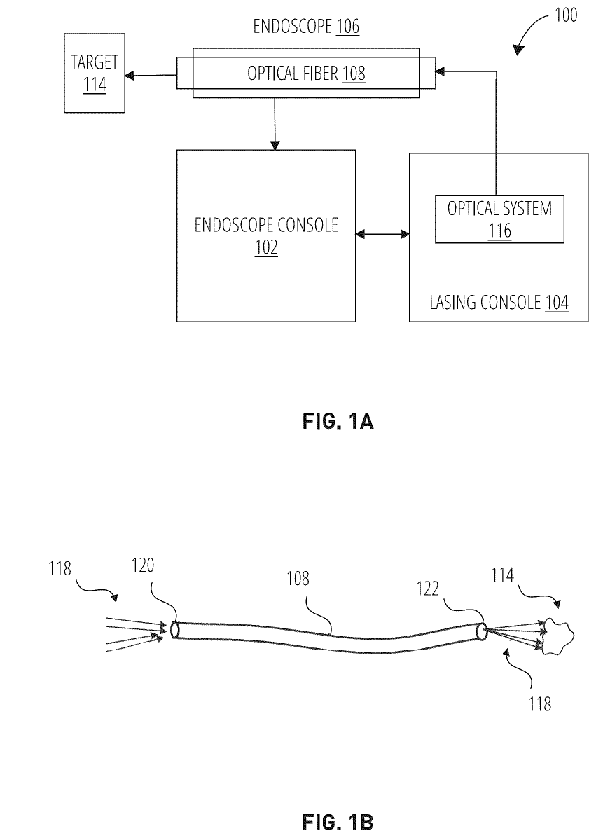

FIG. 1A to FIG. 1D show an exemplary lithotripsy system 100 configured to mitigate interference in image acquisition. Lithotripsy system 100 can comprise an endoscope console 102 and a lasing console 104, coupled to an endoscope 106 and an optical fiber 108, respectively. The endoscope 106 can be any of a variety of “scopes” such as, for example, a ureteroscope, a colonoscope, a bronchoscope, or the like. The endoscope 106 can include one or more working channels in which the optical fiber 108 can be inserted. Further, endoscope 106 can include a camera 110 and a lens 112 (see FIG. 1C and FIG. 1D) of which an image of a target 114 can be acquired. In some embodiments, the target 114 may be a tissue, a stone, a tumor, a cyst, and the like, within a subject, which is to be treated, ablated, or destroyed. In some embodiments, the subject may be a human being or an animal. It is to be appreciated that a variety of different types of imaging endoscopes exit. For example, some endoscopes include a camera on the distal end (e.g., as shown in FIG. 1). However, other cameras include an optical channel (e.g., an optical waveguide, a fiber bundle, or the like) coupled to an external camera or a camera on the proximal portion of the endoscope. Examples are not limited in this context. Further, endoscope 106 can include an illumination source 128 (e.g., an LED, an optical channel coupled to at its proximal end, or the like). The illumination source 128 can be activated to illuminate the environment in which the endoscope distal end is disposed. It will be appreciated that such environment often lacks ambient light so illumination source 128 is provided to illuminate the environment.

Lasing console 104 can comprise an optical system 116 arranged to generate incident light 118 which can be directed to target 114 via optical fiber 108. As depicted more fully in FIG. 1B, the optical fiber 108 comprises a proximal end 120 and a distal end 122. The proximal end 120 is the end of the optical fiber 108 through which light beams enter while the distal end 122 is the end of the optical fiber 108 through which light beams are emitted and via which light beams can be directed onto the target 114. For example, this figure depicts incident light 118 entering the optical fiber 108 at the proximal end 120, propagating through length of the optical fiber 108, exiting the optical fiber 108 at the distal end 122, and being incident on the target 114 from the distal end 122 of the optical fiber 108.

Although not depicted here, optical system 116 can comprise one or more laser light sources and various optics arranged to generate incident light 118 and couple incident light 118 to optical fiber 108. As an example, the laser light sources may include, but are not limited to, solid-state lasers, gas lasers, diode lasers, and fiber lasers. The light beams may include one or more of an aiming beam, a treatment beam, and any other beam transmitted through the optical fiber 108. In various embodiments, an aiming beam may include a light beam of low intensity that is transmitted through the optical fiber 108 to illuminate or highlight the target 114 while a treatment beam may include a light beam of high intensity that is transmitted through the optical fiber 108 to treat the target 114. The various optics within optical system 116 can include, but are not limited to, one or more polarizers, beam splitters, beam combiners, light detector, wavelength division multiplexers, collimators, circulators, and/or lenses. The laser light sources of optical system 116 can comprise a Thulium fiber laser, a Holmium laser, or other types of laser light sources.

Although endoscope console 102 and lasing console 104 are depicted as separate consoles in FIG. 1A, it is to be appreciated that an embodiment could be implemented where endoscope console 102 and lasing console 104 are combined into a single console, for example, sharing many computing components (e.g., processor, memory, display, controls, etc.). However, for purposes of clarity, the disclosure describes examples where endoscope console 102 and lasing console 104 are separate.

Turning more particularly to FIG. 1C and FIG. 1D, camera 110 and lens 112 of endoscope 106 are depicted. As noted above, lens 112 is configured to provide a focal length 124a for camera 110 of a specific distance. However, when vapor bubble 126 is formed (e.g., due to laser energy emitted from optical fiber 108), the vapor bubble 126 acts as another optical element and changes the focal length 124a to a different focal length 124b due to the different index of refraction of the vapor bubble 126, in addition to causing distortion due to irregularity of the vapor bubble.

In addition to interference caused by the focal length 124a changing to focal length 124b due to vapor bubble 126, laser energy interacting with the tissue (e.g., prostate tissue, stone, etc.) can cause additional optical effects that impact the quality of the acquired images. For example, in some cases a short-lived plasma volume can be created when a laser beam is incident on a stone, which causes an extremely bright flash. While short lived, this bright flash will interfere with the image acquisition of the camera 110. As a specific example, effects such as brightness saturation and/or charge-coupled device (CCD) vertical streaking can be captured by camera 110 thereby degrading the quality of the acquired image frame. Often, such effects will degrade the quality of the image frame down to a level that is unusable to a physician.

Lasers often used in endoscopic procedures typically operate at frequencies comparable to the frame rate of the endoscope camera. As such, light from the laser can create significant interference resulting in degradation of the video quality. Further, it will be appreciated that for typical laser-based treatments in endoscopic applications (e.g., for urology, or the like) the laser wavelength is selected so that it has a high absorption coefficient in water. This is done for providing a margin of safety so that laser energy fired in a direction that misses the target will not travel too far before it is absorbed by the liquid in the treatment environment, and thus will not damage other tissue or organs. High water absorption also serves as a means for acting on the target tissue, which usually contains high amount of water. A side effect of the high absorption coefficient in water, is that a vapor bubble is created in the liquid medium when the laser energy is delivered. The vapor inside the bubble has a different refraction index than liquid water. Therefore, the vapor bubble effectively creates an optical element in the water, which lasts for the duration of the bubble.

Endoscopic camera optics are designed to be focused at a typical operating distance, while operating in water. Adding an additional “lens” between the camera and the target (e.g., due to the vapor bubble) causes defocusing and/or distortion of the image, resulting in blurring of the captured image.

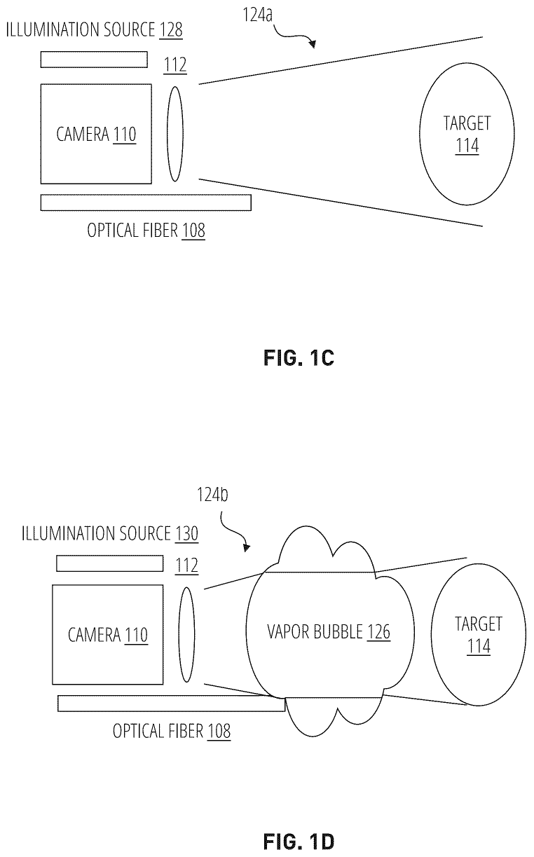

All the above effects can result in blurring of the image captured by the camera 110. For example, FIG. 2A illustrates an endoscopic image 200a, which could be captured by camera 110 of endoscope 106 when a vapor bubble 126 is not extant. As can be seen, the endoscopic image 200a is relatively clear and/or free from distortions or blurring.

FIG. 2B illustrates an endoscopic image 200b, which could be captured by camera 110 of endoscope 106 when a vapor bubble 126 is extant. Unlike endoscopic image 200a, endoscopic image 200b is blurry due to the change in effective focusing distance (e.g., focusing distance 124a to 124b) and distortion due to the vapor bubble 126. It is noted that images 200a and 200b correspond to a frame and a next subsequent frame that immediately follows it. As can be seen, the distortion of the image due to the vapor bubble 126 is instantaneous.

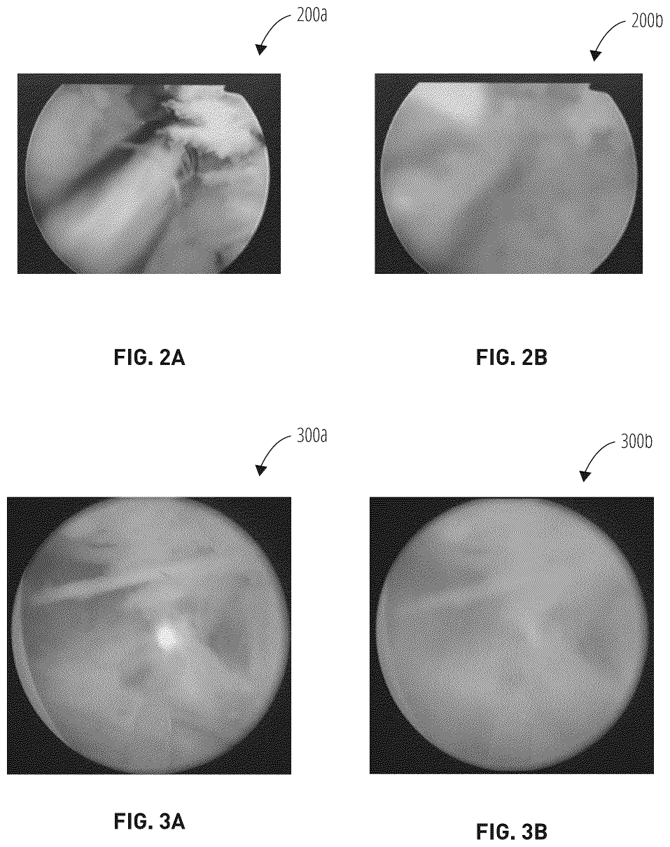

FIG. 3A illustrates another example endoscopic image 300a, which could be captured by camera 110 of endoscope 106 when a vapor bubble 126 is not extant. Like endoscopic image 200a, endoscopic image 300a is relatively clear and/or free from distortions or blurring.

FIG. 3B illustrates an endoscopic image 300b, which could be captured by camera 110 of endoscope 106 when a vapor bubble 126 is extant. Unlike endoscopic image 300a, endoscopic image 300b is blurry due to the change in effective focusing distance (e.g., focusing distance 124a to 124b) and distortion due to the vapor bubble 126.

The present disclosure describes a number of example implementations to mitigate this type of interference, or blurring of the endoscope images resulting from either the change in focal length of the camera 110 when the vapor bubble 126 is extant and/or flashes of light resulting from plasma volumes.

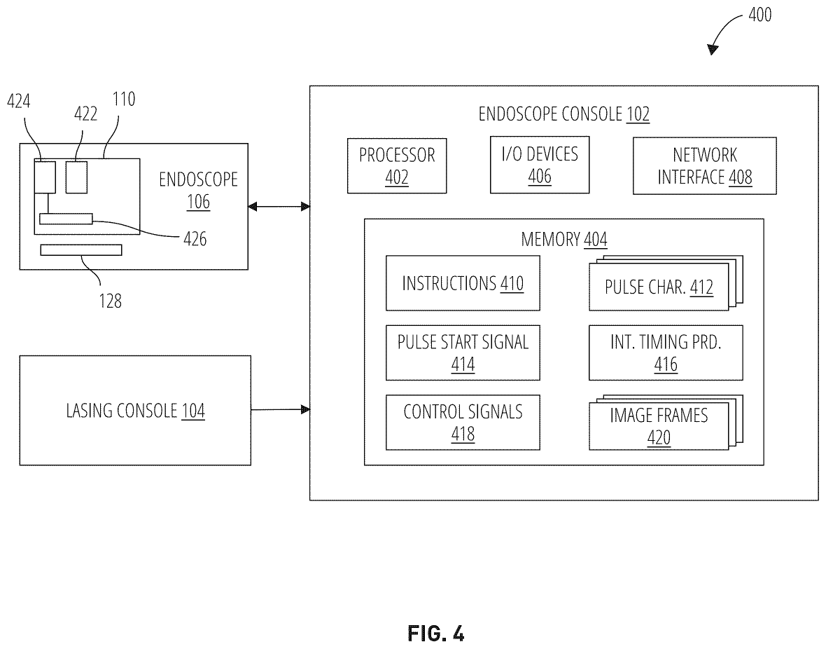

FIG. 4 illustrates an endoscope system 400, in accordance with non-limiting examples of the present disclosure. In general, endoscope system 400 is a system for viewing an image with an endoscope that is configured to reduce interference from lasing. Endoscope system 400 includes endoscope console 102 arranged to be coupled to lasing console 104 and endoscope 106. In general, during operation, endoscope console 102 is arranged to receiving information about laser energy (e.g., laser beams, pulses of laser light, or the like) generated or to be generated by lasing console 104 and to synchronize capturing images from camera 110 in endoscope 106 using the received information about the laser energy to reduce interference with the captured images.

Endoscope console 102 can be any of a variety of computing devices. With some embodiments, endoscope console 102 can be a workstation, server, laptop, or tablet communicatively coupled to endoscope 106 and lasing console 104. Endoscope console 102 can include processor 402, memory 404, input and/or output (I/O) devices 406, and network interface 408. The processor 402 may include circuitry or processor logic, such as, for example, any of a variety of commercial processors. In some examples, processor 402 may include multiple processors, a multi-threaded processor, a multi-core processor (whether the multiple cores coexist on the same or separate dies), and/or a multi-processor architecture of some other variety by which multiple physically separate processors are in some way linked. Additionally, in some examples, the processor 402 may include graphics processing portions and may include dedicated memory, multiple-threaded processing and/or some other parallel processing capability. In some examples, the processor 402 may be an application specific integrated circuit (ASIC) or a field programmable integrated circuit (FPGA).

The memory 404 may include logic, a portion of which includes arrays of integrated circuits, forming non-volatile memory to persistently store data or a combination of non-volatile memory and volatile memory. It is to be appreciated, that the memory 404 may be based on any of a variety of technologies. In particular, the arrays of integrated circuits included in memory 120 may be arranged to form one or more types of memory, such as, for example, dynamic random-access memory (DRAM), NAND memory, NOR memory, or the like.

I/O devices 406 can be any of a variety of devices to receive input and/or provide output. For example, I/O devices 406 can include, a keyboard, a mouse, a joystick, a foot pedal, a display, a touch enabled display, a haptic feedback device, an LED, or the like.

Network interface 408 can include logic and/or features to support a communication interface. For example, network interface 408 may include one or more interfaces that operate according to various communication protocols or standards to communicate over direct or network communication links. Direct communications may occur via use of communication protocols or standards described in one or more industry standards (including progenies and variants). For example, network interface 408 may facilitate communication over a bus, such as, for example, peripheral component interconnect express (PCIe), non-volatile memory express (NVMe), universal serial bus (USB), system management bus (SMBus), SAS (e.g., serial attached small computer system interface (SCSI)) interfaces, serial AT attachment (SATA) interfaces, or the like. Additionally, network interface 408 can include logic and/or features to enable communication over a variety of wired or wireless network standards (e.g., 802.11 communication standards). For example, network interface 408 may be arranged to support wired communication protocols or standards, such as, Ethernet, or the like. As another example, network interface 408 may be arranged to support wireless communication protocols or standards, such as, for example, Wi-Fi, Bluetooth, ZigBee, LTE, 5G, or the like.

Memory 404 can include instructions 410, laser pulse characteristics 412, laser pulse start signal 414, camera integration timing period 416, control signals 418, and image frames 420. In general, during operation, processor 402 can execute instructions 410 to cause endoscope console 102 to receive laser pulse characteristics 412 and laser pulse start signal 414 from lasing console 104. Laser pulse characteristics 412 can comprise an indication of the pulse repetition rate (e.g., laser pulse frequency, or the like) as well as the duration of the pulse (e.g., pulse width, or the like).

Additionally, processor 402 can execute instructions 410 to generate camera integration timing period 416 from laser pulse characteristics 412 and laser pulse start signal 414. As will be appreciated digital cameras, like camera 110, operate by accumulating charge (or integrating charge) responsive to incident light at points (e.g., pixel cells, frame lines, etc.) in a frame and then “reading out” the charge to a memory location. More specifically, the overall “image acquisition” process for a digital camera includes 4 main parts: (1) clear or reset where each pixel cell is cleared of any accumulated charge, (2) integrate (e.g., accumulate charge) in the pixel cells responsive to incident light, (3) transfer the charge out of the cells, and (4) read out pixel data to a memory storage location. Accordingly, interference from lasing (e.g., either the laser pulse itself or plasma volumes) will only affect the captured image when the interference and integration of charge overlap.

Camera 110 can include a camera sensor 422, a shutter 424, and shutter control circuitry and mechanism 426. During operation, the shutter control circuitry and mechanism 426 can control the opening and closing of shutter 424 to allow or block light from being incident on the camera sensor 422. For example, during the integration phase shutter control circuitry and mechanism 426 can cause shutter 424 to open to allow incident light to imping on camera sensor 422 such that charge can accumulate in the lines (e.g., capacitors, cells, or the like) of the camera sensor 422. It is to be appreciated that many modern cameras may include either a mechanical shutter or an electronic shutter. Although the mechanics or electronics of camera shutter systems is beyond the scope of this disclosure and the concepts described herein to gate the integration phase of a camera sensor can be applied to either mechanical or electronic shutter systems, a brief description of electronic shutter systems is described herein.

The present disclosure provides to generate camera integration timing period 416 comprising an indication of when to integrate or accumulate charge in the pixel cells (or pixel lines) so that the integration will take place outside times when interference is present. Processor 402 can execute instructions 410 to generate control signals 418 from camera integration timing period 416 and communicate control signals 418 to endoscope 106 (and particularly camera 110) such that the image frames 420 can be captured by camera 110 and received by endoscope console 102 where interference from laser energy emitted by lasing console 104 is mitigated.

It will be appreciated that camera 110 can be implemented with a variety of different camera shutter sensors, such as, for example, a global shutter sensor or a rolling shutter sensor. It will further be appreciated that the difference between a global shutter sensor and a rolling shutter sensor is that integration for all cells is simultaneous while memory read out is staggered whereas integration and memory readout is staggered for rolling shutter sensors. Accordingly, if interference overlaps the integration period for a global shutter sensor, the interference will affect all sensor lines while it will only some of the sensor lines for a rolling shutter sensor.

Lasers can be operated at repetition rates similar, or identical to, camera frame rates. As such, many synchronization effects that cause impacts on a time scale beyond interference with a particular frame can result. For example, for a global shutter camera operating at 30 Hertz (Hz) and a laser operating at 30 Hz, the frequencies could be synchronized by chance so that lasing always occurs during the camera readout phase and no interference is registered in the image frames. In another example, the frequencies could be synchronized so that lasing always occurs during the integrating phase, and as such, all image frames will have interference. In yet another example, for a global shutter camera operating at 30 Hz and a laser operating at 60 Hz all image frames will have interference from at least 1 laser pulse. With yet another example, for a global shutter camera operating at 30 Hz and a laser operating a 5 Hz, every 6th image frame may have interference, which may manifest as a “flickering” of the video signal. Still, in other examples, for a rolling shutter camera operating at 30 Hz and a laser operating at 30 Hz, some of the image frame lines will always have interference creating a pattern of repeating interference on the resulting video. Still in another example, for a rolling shutter camera operating at 30 Hz and a laser operating at “about” 30 Hz (e.g., due to small differences in clock accuracy, timing circuitry, or the like) the interference will slowly “drift” across the image frames and manifest and drifting interference on the video.

To that end, the present disclosure provides the endoscope system 400 configured to synchronize image capture and particularly integration or accumulation of charge in the image sensor of camera 110 with lasing of lasing console 104 based on a variety of different techniques, which can be implemented by endoscope system 400 based on the type of camera sensor (e.g., global shutter, rolling shutter), the frequency of the camera 110, and/or the frequency of the lasing console 104.

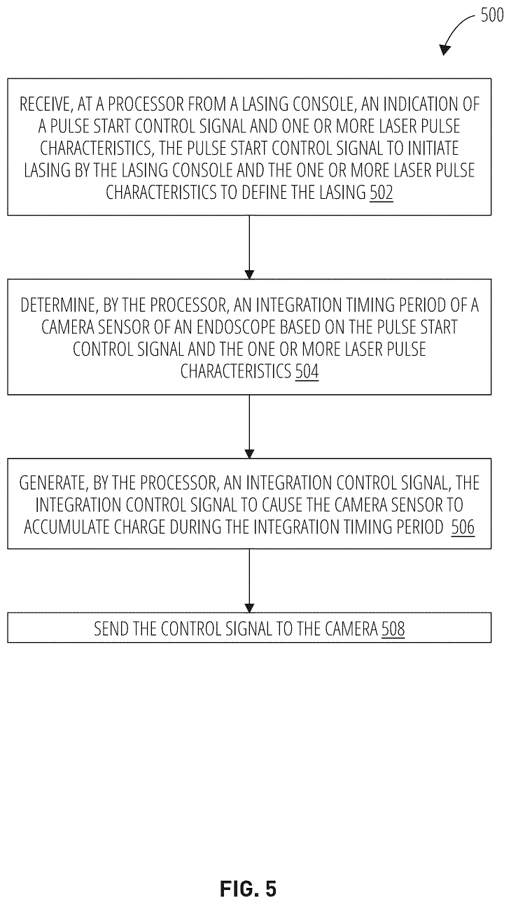

FIG. 5 illustrates a method 500 to synchronize the integration phase of camera with a lasing console. For example, endoscope console 102 can implement method 500 to synchronize the integration phase of camera 110 with lasing by lasing console 104. For example, processor 402 can execute instructions 410 to cause endoscope console 102 to implement method 500.

Method 500 can begin at block 502. At block 502 “receive, from a lasing console, an indication of a pulse start control signal and one or more laser pulse characteristics, the pulse start control signal to initiate lasing by the lasing console and the one or more laser pulse characteristics to define the lasing” a pulse start control signal and one or more laser pulse characteristics can be received from a lasing console. The pulse start control signal can define an initiation of lasing by the lasing console while the one or more laser pulse characteristics can define the lasing. For example, processor 402 can execute instructions 410 to receive laser pulse start signal 414 and laser pulse characteristics 412. As detailed above laser pulse start signal 414 can correspond to a signal that defines the initiation of lasing by lasing console 104 while laser pulse characteristics 412 can define the lasing itself (e.g., pulse width, frequency, etc.)

Continuing to block 504 “determine, by the processor, an integration timing period of a camera sensor of an endoscope based on the pulse start control signal and the one or more laser pulse characteristics” an integration timing period for a camera sensor can be determined based on the pulse start control signal and the one or more laser pulse characteristics. For example, processor 402 can execute instructions 410 to determine camera integration timing period 416 based on laser pulse start signal 414 and laser pulse characteristics 412 where camera integration timing period 416 where camera integration timing period 416 defines a period over which integration is to take place. It is to be appreciated that the present disclosure provides to determine the integration timing period in a variety of manners, which can vary based on the type of shutter system (e.g., global shutter, rolling shutter, etc.) However, all methods and devices described herein provide to determine the integration timing signal based on the laser pulse to be generated by the lasing console 104. For example, processor 402 can execute instructions 410 to determine a period in which to open shutter 424 such that camera sensor 422 can accumulate charge when the lasing console 104 is not lasing. In many embodiments, this will result in a shortened integration phase than conventional camera operation. This will be described in greater detail below.

Continuing to block 506 “generate, by the processor, an integration control signal, the integration control signal to cause the camera sensor to accumulate charge during the integration timing period” an integration control signal can be generated where the integration control signal is configured to cause a camera sensor to accumulate charge during a period specified by the control signal. For example, processor 402 can execute instructions 410 to generate control signals 418 where control signals 418 can cause shutter 424 to open during the integration timing period (e.g., camera integration timing period 416, or the like) such that camera sensor 422 can accumulate charge during the integration timing period.

Continuing to block 508 “send the control signal to the camera” the control signal can be sent to the camera. For example, processor 402 can execute instructions 410 to send the control signals 418 to the camera 110 or rather, to shutter control circuitry and mechanism 426 which can cause shutter 424 to open and/or close based on camera integration timing period 416. As used herein, the phrases open shutter 424 and close shutter 424 could apply to either opening and closing a mechanical shutter or could apply to starting and stopping an integration phase of an electronic shutter. For example, as noted above shutter 424 may not be a physical mechanical shutter but instead may be an electronic shutter arranged to accumulate charge in sensor lines and read the accumulated charge from the sensor lines. The phrase open shutter 424 could in such an example mean initiating an accumulation of charge in the sensor lines while the phrase close shutter 424 could mean stopping the accumulation of charge. The method 500 can further include blocks (not shown) where information elements comprising indications of frame data can be received from the camera, processed, and displayed on a display. For example, processor 402 can execute instructions 410 to receive image frames 420 from camera 110 (e.g., via a memory readout process, or the like) and can process the image frames 420 to generate images to display on a display coupled to the endoscope system 400. In particular, the method 500 can be implemented in real time, or rather, during a procedure, such that capturing images by camera 110 of endoscope 106 can be synchronized with lasing by lasing console 104 to reduce and/or mitigate interference manifest in the captured images.

As noted, method 500 can be provided to generate the integration timing period (e.g., camera integration timing period 416) in a variety of different ways. With some embodiments, where camera 110 is a global shutter style camera, processor 402 can execute instructions 410 to determine reduce a duration of the maximum integration period by a duration of the interference to determine the integration timing period. Processor 402 can execute instructions 410 to determine a duration of interference based on the laser pulse characteristics 412 and laser pulse start signal 414. For example, for a 30 Hz laser pulse with a pulse width of 1 millisecond (ms) processor 402 can execute instructions 410 to determine that the duration of the interference is 1 ms, which repeats every 33 ms. Further, for a 30 Hz camera, processor 402 can determine that the maximum duration of the integration timing period can be 33 ms. Given the duration of the interference (e.g., based on laser pulse characteristics 412 and laser pulse start signal 414) and the maximum duration of the integration timing period (e.g., based on settings for camera 110), processor 402 can execute instructions 410 to determine camera integration timing period 416 by subtracting the duration of the interference from the maximum duration of the integration timing period (e.g., 33 ms minus 1 ms in the example above). As integration for all memory lines is done in parallel in a global shutter camera, accumulation of charge for all lines or cells will be shortened. Further, processor 402 can execute instructions 410 to generate control signals 418 such that the shutter 424 of camera 110 will open for the determined camera integration timing period 416 outside the time the laser beam is activated (e.g., based on laser pulse characteristics 412).

In other embodiments, where camera 110 is a rolling shutter style camera, processor 402 can execute instructions 410 to determine camera integration timing period 416 in a similar manner to that described for a global shutter camera above. However, as integration is done in a rolling (e.g., often overlapping) fashion, accumulation of charge for certain lines will be shortened, or paused, while integration for other cells will be unaffected. However, as memory readout is done in parallel to integration, shortening the integration phase for some lines may result in a delay in memory readout for those lines. As such, larger memory buffers and/or memory processing circuitry on the receiving hardware side may be needed to address the delay and reduce the chance of non-contiguous frames being generated.

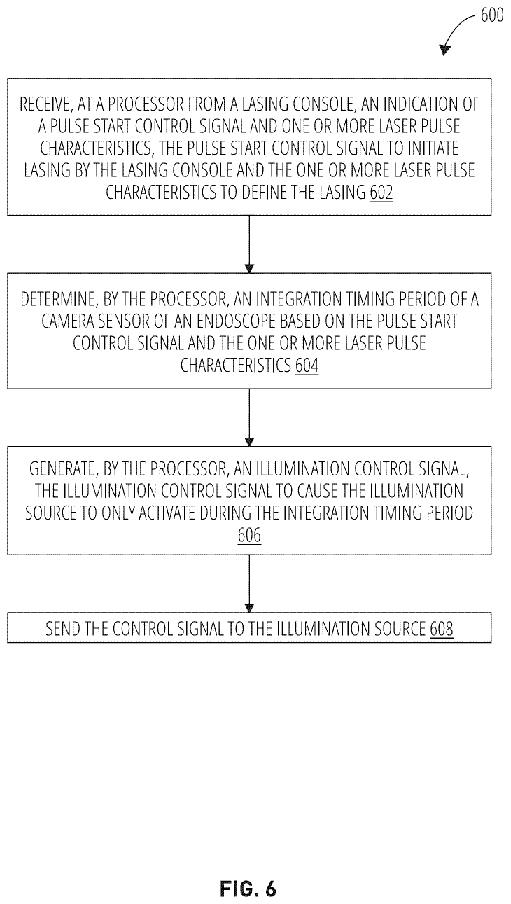

FIG. 6 illustrates a method 600 to synchronize the illumination of an environment (and thus the integration phase of a camera) with a lasing console. For example, endoscope console 102 can implement method 600 to synchronize the activation of illumination source 128 with lasing by lasing console 104. For example, processor 402 can execute instructions 410 to cause endoscope console 102 to implement method 600.

Method 600 can begin at block 602. At block 602 “receive, from a lasing console, an indication of a pulse start control signal and one or more laser pulse characteristics, the pulse start control signal to initiate lasing by the lasing console and the one or more laser pulse characteristics to define the lasing” a pulse start control signal and one or more laser pulse characteristics can be received from a lasing console. The pulse start control signal can define an initiation of lasing by the lasing console while the one or more laser pulse characteristics can define the lasing. For example, processor 402 can execute instructions 410 to receive laser pulse start signal 414 and laser pulse characteristics 412. As detailed above laser pulse start signal 414 can correspond to a signal that defines the initiation of lasing by lasing console 104 while laser pulse characteristics 412 can define the lasing itself (e.g., pulse width, frequency, etc.)

Continuing to block 604 “determine, by the processor, an integration timing period of a camera sensor of an endoscope based on the pulse start control signal and the one or more laser pulse characteristics” an integration timing period for a camera sensor can be determined based on the pulse start control signal and the one or more laser pulse characteristics. For example, processor 402 can execute instructions 410 to determine camera integration timing period 416 based on laser pulse start signal 414 and laser pulse characteristics 412 where camera integration timing period 416 where camera integration timing period 416 defines a period over which integration is to take place. It is to be appreciated that the present disclosure provides to determine the integration timing period in a variety of manners, which can vary based on the type of shutter system (e.g., global shutter, rolling shutter, etc.) However, all methods and devices described herein provide to determine the integration timing signal based on the laser pulse to be generated by the lasing console 104. For example, processor 402 can execute instructions 410 to determine a period in which to open shutter 424 such that camera sensor 422 can accumulate charge when the lasing console 104 is not lasing. In many embodiments, this will result in a shortened integration phase than conventional camera operation. This will be described in greater detail below.

Continuing to block 606 “generate, by the processor, an illumination control signal, the illumination control signal to cause the illumination source to only activate during the integration timing period” an illumination control signal can be generated where the illumination control signal is configured to cause an illumination source to activate during a period specified by the control signal. For example, processor 402 can execute instructions 410 to generate control signals 418 where control signals 418 can cause illumination source 128 to activate during the integration timing period (e.g., camera integration timing period 416, or the like) such that even when camera sensor 422 accumulates charge, accent lighting (e.g., non-ambient light) will only be provided during the integration timing period.

Continuing to block 608 “send the control signal to the illumination source” the control signal can be sent to the illumination source. For example, processor 402 can execute instructions 410 to send the control signals 418 to the illumination source 128. The method 600 can further include blocks (not shown) where information elements comprising indications of frame data can be received from the camera, processed, and displayed on a display. For example, processor 402 can execute instructions 410 to receive image frames 420 from camera 110 (e.g., via a memory readout process, or the like) and can process the image frames 420 to generate images to display on a display coupled to the endoscope system 400. In particular, the method 600 can be implemented in real time, or rather, during a procedure, such that capturing images by camera 110 of endoscope 106 can be synchronized with lasing by lasing console 104 to reduce and/or mitigate interference manifest in the captured images.

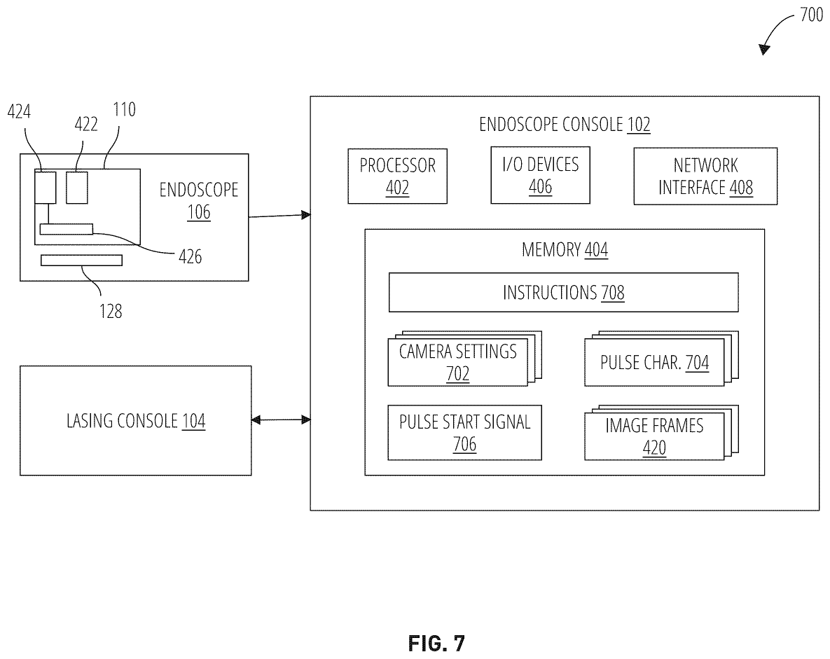

FIG. 7 illustrates an endoscope system 700, in accordance with non-limiting examples of the present disclosure. In general, endoscope system 700 is a system for viewing an image with an endoscope that is configured to reduce interference from lasing. Endoscope system 700 includes many of the same components described above with respect to endoscope system 400 and FIG. 4. The difference being that endoscope system 700 is configured to change the shutter settings of camera 110 to reduce interference while endoscope system 700 is configured to change the settings of the lasing console 104 to reduce interference.

To that end, memory 404 can include instructions 708, camera settings 702, laser pulse characteristics 704, laser pulse start signal 706, and image frames 420. In general, during operation, processor 402 can execute instructions 708 to cause endoscope console 102 to receive camera settings 702 from camera 110. Camera settings 702 can comprise an indication of the frequency of camera 110 operation, the shutter type, and/or memory readout settings, or the like. Additionally, processor 402 can execute instructions 708 to generate laser pulse characteristics 704 and laser pulse start signal 706 from camera settings 702. For example, processor 402 can execute instructions 410 to determine period where integration is not taking place based on camera settings 702 (e.g., period of memory readout for global shutter type cameras, vertical blanking periods, or the like) and determine a laser pulse characteristics 704 (e.g., pulse repetition rate, pulse duration, etc.) and laser pulse start signal 706 such that lasing by lasing console 104 takes place during these periods (e.g., outside the integration phase). Processor 402 can further execute instructions 708 to send information elements comprising indications of laser pulse characteristics 704 and laser pulse start signal 706 to lasing console 104 such that lasing console 104 can generate pulses of light based on laser pulse characteristics 704 and laser pulse start signal 706.

It will be appreciated that the laser pulse characteristics 704 will need to be synchronized with the frequency or frame rate of the camera 110. For example, where camera 110 has a frame rate of 30 Hz, laser pulse characteristics 704 can comprise a pulse rate of 30 Hz, 15 Hz, 10 Hz, 6 Hz, 5 Hz, or 3 Hz, but cannot exceed 30 Hz. As such, endoscope system 700 may not be practical for all laser system applications (e.g., those operating at 80 Hz, or the like).

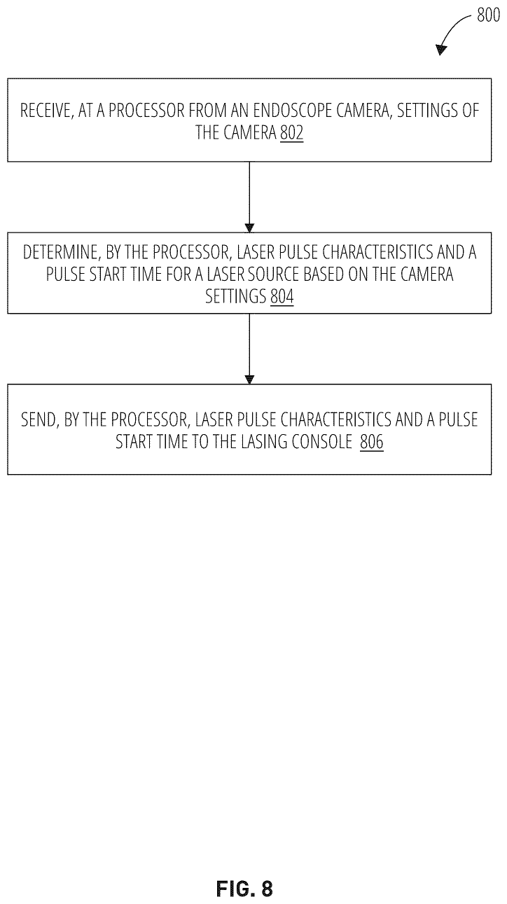

FIG. 8 illustrates a method 800 to synchronize lasing of a laser source with the integration phase of an endoscope camera. For example, endoscope console 102 can implement method 800 to synchronize lasing by lasing console 104 with the integration phase of camera 110. For example, processor 402 can execute instructions 708 to cause endoscope console 102 to implement method 800.

Method 800 can begin at block 802. At block 802 “receive, at a processor from an endoscope camera, settings of the camera” settings for a camera can be received from an endoscope camera. For example, processor 402 can execute instructions 708 to receive camera settings 702 from camera 110. As detailed above camera settings 702 can correspond to the shutter type, frame rate, memory readout settings, timing of a vertical blanking interval, or the like.

Continuing to block 804 “determine, by the processor, laser pulse characteristics and a pulse start time for a laser source” laser pulse characteristics and a pulse start time for a laser source can be determined based on the camera settings. For example, processor 402 can execute instructions 708 to determine laser pulse characteristics 704 and laser pulse start signal 706 based on camera settings 702. As described above, laser pulse characteristics 704 can include a pulse repetition rate and a pulse duration. The pulse repetition rate will be a factor of the frame rate of the camera 110, but will not exceed the frame rate of the camera. The pulse duration will be generated such that given the pulse repetition rate, the laser pulse is generated in a time when integration is not taking place (e.g., during memory readout, during a vertical blanking interval, or the like).

Continuing to block 806 “send, by the processor, laser pulse characteristics and a pulse start time to the lasing console” the determined laser pulse characteristics and pulse start time can be communicated with the laser console such that the laser console can generate laser pulses that occur outside an integration phase of camera 110. For example, processor 402 can execute instructions 708 to send laser pulse characteristics 704 and laser pulse start signal 706 to lasing console 104 and cause lasing console 104 to generate laser pulses based on laser pulse characteristics 704 and laser pulse start signal 706.

Like method 500, method 800 can further include blocks (not shown) where information elements comprising indications of frame data can be received from the camera, processed, and displayed on a display. For example, processor 402 can execute instructions 410 to receive image frames 420 from camera 110 (e.g., via a memory readout process, or the like) and can process the image frames 420 to generate images to display on a display coupled to the endoscope system 400. In particular, the method 800 can be implemented in real time, or rather during a procedure, such that lasing by lasing console 104 can be synchronized with capturing images by camera 110 of endoscope 106 to reduce and/or mitigate interference manifest in the captured images.

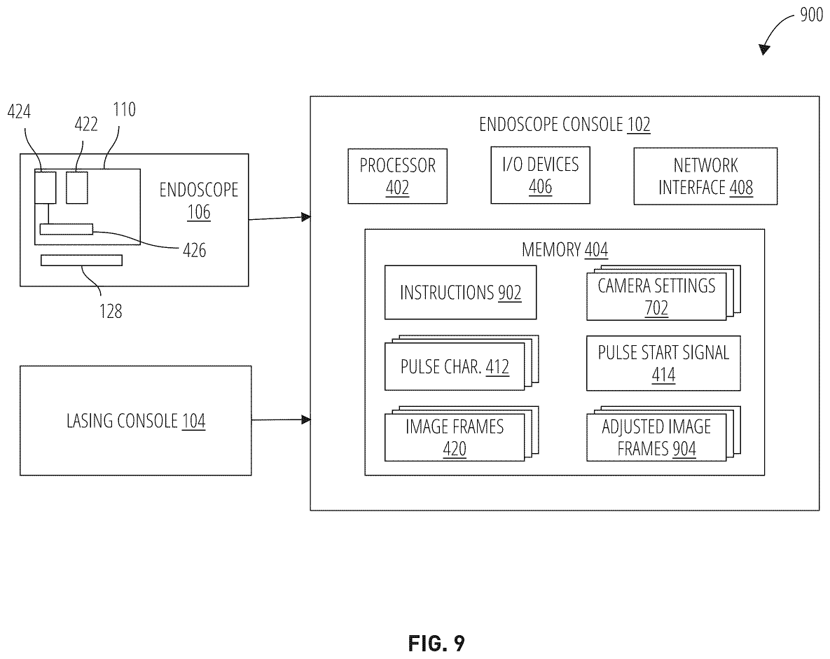

FIG. 9 illustrates an endoscope system 900, in accordance with non-limiting examples of the present disclosure. In general, endoscope system 900 is a system for viewing an image with an endoscope that is configured to reduce interference from lasing. Endoscope system 900 includes many of the same components described above with respect to endoscope system 400 of FIG. 4 and endoscope system 700 of FIG. 7. The difference being that endoscope system 900 is configured to identify frames and/or portions of a frame where interference occurred based on the above detailed camera/laser synchronization details and generate adjusted frames to remove the interference.

To that end, memory 404 can include instructions 902, camera settings 702, laser pulse characteristics 412, laser pulse start signal 414, image frames 420, and adjusted image frames 904. In general, during operation, processor 402 can execute instructions 902 to cause endoscope console 102 to receive camera settings 702 from camera 110. Camera settings 702 can comprise an indication of the frequency of camera 110 operation, the shutter type, and/or memory readout settings, or the like. Additionally, processor 402 can execute instructions 902 to cause endoscope console 102 to receive laser pulse characteristics 412 (e.g., frequency, duration, etc.) and laser pulse start signal 414 from lasing console 104. Further, processor 402 can execute instructions 902 to receive image frames 420 from endoscope 106.

Given camera settings 702, laser pulse characteristics 412, and laser pulse start signal 414, processor 402 can execute instructions 902 to determine frames of image frames 420 and/or portions of a frame in image frames 420 in which interference occurred (e.g., where lasing by lasing console 104 overlapped with an integration phase by camera 110). Processor 402 can execute instructions 902 to replace the identifies frames or portions of frames with a prior one of image frames 420 (or a portion of a prior frame as may be the case).

For example, for a global shutter camera system, processor 402 can execute instructions 902 to determine frames from image frames 420 where lasing by lasing console 104 overlapped with integration by camera 110 and replace the identified frames with the most recent prior frame where lasing by lasing console 104 did not overlap with integration by camera 110. As another example, for a rolling shutter camera system, processor 402 can execute instructions 902 to determine which lines of a frame integration by camera 110 overlapped with lasing by lasing console 104 and replace the lines of the frame with prior lines where integration by camera 110 did not overlap with lasing by lasing console 104.

It is to be appreciated that replacing frames in image frames 420 with prior frames to generate adjusted image frames 904 may cause fast moving objects to appear to have an un-smooth movement in a resulting video generated from adjusted image frames 904. Accordingly, practicality of use for endoscope system 900 may be limited to situations where the scene being imaged by camera 110 of endoscope 106 is changing slowly (e.g., with little change between the current frame and prior frame). Further, endoscope system 900 may be more practical where the repetition rate of the laser pulse generated by lasing console 104 is slower than the frame rate of camera 110, and as such, the interference will not manifest in every frame, and even if it does manifest in every frame, it will not manifest in the same video lines of each frame.

With some embodiments, camera 110 of endoscope 106 can have a frame rate that is higher than a repetition rate of the lasing console 104. Processor 402 can execute instructions 902 to identify frames of image frames (e.g., one or more frames of the several image frames 420, or the like) captured when and lasing console 104 is emitting a laser beam (e.g., resulting in possible interference in the captured image). Processor 402 can execute instructions 902 to generate adjusted image frames 904 by removing the identified image frames from the set of image frames. Said differently, processor 402 can execute instructions 902 to remove the identified image frames from the image frames 420 to form the adjusted image frames 904. Further, processor 402 can execute instructions 902 to recreate and/or render a video stream based on the adjusted image frames 904.

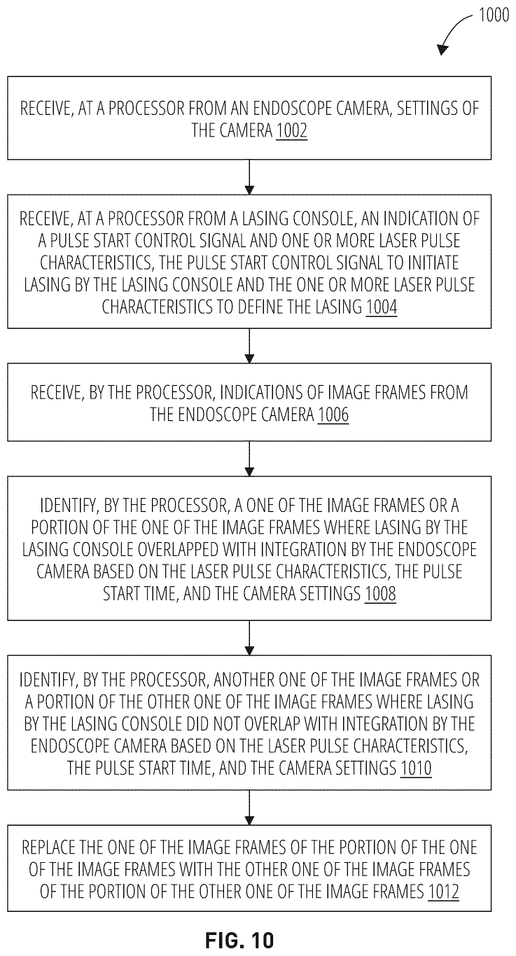

FIG. 10 illustrates a method 1000 to remove interference from image frames captured by an endoscope camera based on synchronizing lasing of a laser source with the integration phase of the endoscope camera. For example, endoscope console 102 can implement method 1000 to generate adjusted or updated frames by replacing a frame (or portions of the frame) where integration of camera 110 of endoscope 106 overlapped with another frame (or portions of the other frame) where lasing by lasing console 104 did not overlap with integration by camera 110 of endoscope 106.

Method 1000 can begin at block 1002. At block 1002 “receive, at a processor from an endoscope camera, settings of the camera” settings for a camera can be received from an endoscope camera. For example, processor 402 can execute instructions 902 to receive camera settings 702 from camera 110. As detailed above camera settings 702 can correspond to the shutter type, frame rate, memory readout settings, timing of a vertical blanking interval, or the like.

Continuing to block 1004 “receive, from a lasing console, an indication of a pulse start control signal and one or more laser pulse characteristics, the pulse start control signal to initiate lasing by the lasing console and the one or more laser pulse characteristics to define the lasing” a pulse start control signal and one or more laser pulse characteristics can be received from a lasing console. The pulse start control signal can define an initiation of lasing by the lasing console while the one or more laser pulse characteristics can define the lasing. For example, processor 402 can execute instructions 902 to receive laser pulse start signal 414 and laser pulse characteristics 412. As detailed above laser pulse start signal 414 can correspond to a signal that defines the initiation of lasing by lasing console 104 while laser pulse characteristics 412 can define the lasing itself (e.g., pulse width, frequency, etc.)

Continuing to block 1006 “receive, by the processor, indications of image frames from the endoscope camera” indication of image frames can be received from the camera of the endoscope. For example, processor 402 can execute instructions 902 to receive image frames 420 from camera 110 of endoscope 106. Continuing to block 1008 “identify, by the processor, a one of the image frames or a portion of the one of the image frames where lasing by the lasing console overlapped with integration by the endoscope camera based on the laser pulse characteristics, the pulse start time, and the camera settings” a frame or portions of the frame of the received frames where integration overlapped with lasing can be identified. For example, processor 402 can execute instructions 902 to identify a frame from image frames 420, or a portion (e.g., lines) of the frame of image frames 420 where integration of the camera 110 of endoscope 106 overlapped with lasing by lasing console 104 based on camera settings 702, laser pulse characteristics 412, and laser pulse start signal 414.

Continuing to block 1010 “identify, by the processor, another one of the image frames or a portion of the other one of the image frames where lasing by the lasing console did not overlap with integration by the endoscope camera based on the laser pulse characteristics, the pulse start time, and the camera settings” another frame, or portions of the other frame, of the received frames where integration did not overlap with lasing can be identified. For example, processor 402 can execute instructions 902 to identify a frame from image frames 420, or a portion (e.g., lines) of the frame of image frames 420 where integration of the camera 110 of endoscope 106 did not overlap with lasing by lasing console 104 based on camera settings 702, laser pulse characteristics 412, and laser pulse start signal 414. It is noted that the frame identified at block 1010 will be prior to the frame identified at block 1008.

Continuing to block 1012 “replace the one of the image frames of the portion of the one of the image frames with the other one of the image frames of the portion of the other one of the image frames” the frame (or portions of the frame) identified at block 1008 can be replaced by the frame (or portions of the frame) identified at block 1010. For example, processor 402 can execute instructions 902 to generate adjusted image frames 904 by replacing the frame (or portions of the frame) from image frames 420 identified as having overlapping integration and lasing with the prior frame (or portions of the prior frame) identified as not having overlapping integration and lasing.

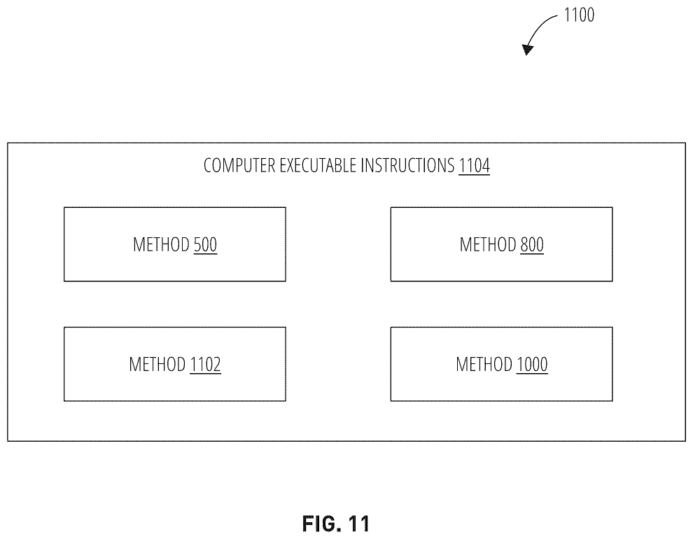

FIG. 11 illustrates computer-readable storage medium 1100. Computer-readable storage medium 1100 may comprise any non-transitory computer-readable storage medium or machine-readable storage medium, such as an optical, magnetic or semiconductor storage medium. In various embodiments, computer-readable storage medium 1100 may comprise an article of manufacture. In some embodiments, computer-readable storage medium 1100 may store computer executable instructions 1102 with which circuitry (e.g., processor 402, or the like) can execute. For example, computer executable instructions 1002 can include instructions to implement operations described with respect to method 500, method 600, method 800, and/or method 1000. Examples of computer-readable storage medium 1100 or machine-readable storage medium may include any tangible media capable of storing electronic data, including volatile memory or non-volatile memory, removable or non-removable memory, erasable or non-erasable memory, writeable or re-writeable memory, and so forth. Examples of computer executable instructions 1102 may include any suitable type of code, such as source code, compiled code, interpreted code, executable code, static code, dynamic code, object-oriented code, visual code, and the like.

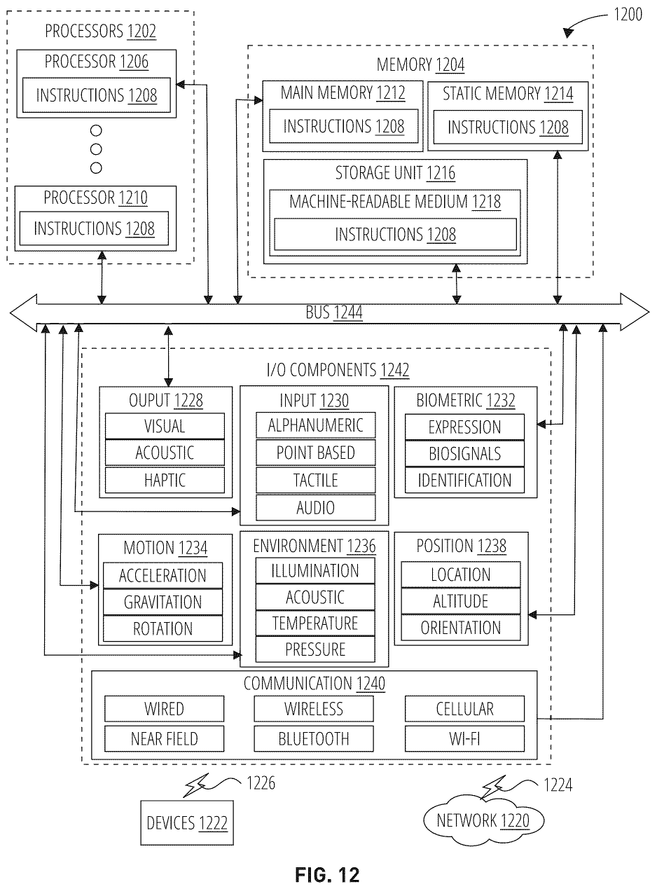

FIG. 12 illustrates a diagrammatic representation of a machine 1200 in the form of a computer system within which a set of instructions may be executed for causing the machine to perform any one or more of the methodologies discussed herein. More specifically, FIG. 12 shows a diagrammatic representation of the machine 1200 in the example form of a computer system, within which instructions 1208 (e.g., software, a program, an application, an applet, an app, or other executable code) for causing the machine 1200 to perform any one or more of the methodologies discussed herein may be executed. For example, the instructions 1208 may cause the machine 1200 to execute method 500 of FIG. 5, method 600 of FIG. 6, method 800 of FIG. 8, and/or method 1000 of FIG. 10, or the like. More generally, the instructions 1208 may cause the machine 1200 to synchronize lasing by a laser source with integration by an endoscope camera, synchronize integration by an endoscope camera with lasing by a laser source, or replace frames in captured images based on the timing of lasing by a laser source and integration by an endoscope camera.

The instructions 1208 transform the general, non-programmed machine 1200 into a particular machine 1200 programmed to carry out the described and illustrated functions in a specific manner. In alternative embodiments, the machine 1200 operates as a standalone device or may be coupled (e.g., networked) to other machines. In a networked deployment, the machine 1200 may operate in the capacity of a server machine or a client machine in a server-client network environment, or as a peer machine in a peer-to-peer (or distributed) network environment. The machine 1200 may comprise, but not be limited to, a server computer, a client computer, a personal computer (PC), a tablet computer, a laptop computer, a netbook, a set-top box (STB), a PDA, an entertainment media system, a cellular telephone, a smart phone, a mobile device, a wearable device (e.g., a smart watch), a smart home device (e.g., a smart appliance), other smart devices, a web appliance, a network router, a network switch, a network bridge, or any machine capable of executing the instructions 1208, sequentially or otherwise, that specify actions to be taken by the machine 1200. Further, while only a single machine 1200 is illustrated, the term “machine” shall also be taken to include a collection of machines 200 that individually or jointly execute the instructions 1208 to perform any one or more of the methodologies discussed herein.

The machine 1200 may include processors 1202, memory 1204, and I/O components 1242, which may be configured to communicate with each other such as via a bus 1244. In an example embodiment, the processors 1202 (e.g., a Central Processing Unit (CPU), a Reduced Instruction Set Computing (RISC) processor, a Complex Instruction Set Computing (CISC) processor, a Graphics Processing Unit (GPU), a Digital Signal Processor (DSP), an ASIC, a Radio-Frequency Integrated Circuit (RFIC), another processor, or any suitable combination thereof) may include, for example, a processor 1206 and a processor 1210 that may execute the instructions 1208. The term “processor” is intended to include multi-core processors that may comprise two or more independent processors (sometimes referred to as “cores”) that may execute instructions contemporaneously. Although FIG. 12 shows multiple processors 1202, the machine 1200 may include a single processor with a single core, a single processor with multiple cores (e.g., a multi-core processor), multiple processors with a single core, multiple processors with multiples cores, or any combination thereof.

The memory 1204 may include a main memory 1212, a static memory 1214, and a storage unit 1216, both accessible to the processors 1202 such as via the bus 1244. The main memory 1204, the static memory 1214, and storage unit 1216 store the instructions 1208 embodying any one or more of the methodologies or functions described herein. The instructions 1208 may also reside, completely or partially, within the main memory 1212, within the static memory 1214, within machine-readable medium 1218 within the storage unit 1216, within at least one of the processors 1202 (e.g., within the processor's cache memory), or any suitable combination thereof, during execution thereof by the machine 1200.

The I/O components 1242 may include a wide variety of components to receive input, provide output, produce output, transmit information, exchange information, capture measurements, and so on. The specific I/O components 1242 that are included in a particular machine will depend on the type of machine. For example, portable machines such as mobile phones will likely include a touch input device or other such input mechanisms, while a headless server machine will likely not include such a touch input device. It will be appreciated that the I/O components 1242 may include many other components that are not shown in FIG. 12. The I/O components 1242 are grouped according to functionality merely for simplifying the following discussion and the grouping is in no way limiting. In various example embodiments, the I/O components 1242 may include output components 1228 and input components 1230. The output components 1228 may include visual components (e.g., a display such as a plasma display panel (PDP), a light emitting diode (LED) display, a liquid crystal display (LCD), a projector, or a cathode ray tube (CRT)), acoustic components (e.g., speakers), haptic components (e.g., a vibratory motor, resistance mechanisms), other signal generators, and so forth. The input components 1230 may include alphanumeric input components (e.g., a keyboard, a touch screen configured to receive alphanumeric input, a photo-optical keyboard, or other alphanumeric input components), point-based input components (e.g., a mouse, a touchpad, a trackball, a joystick, a motion sensor, or another pointing instrument), tactile input components (e.g., a physical button, a touch screen that provides location and/or force of touches or touch gestures, or other tactile input components), audio input components (e.g., a microphone), and the like.

In further example embodiments, the I/O components 1242 may include biometric components 1232, motion components 1234, environmental components 1236, or position components 1238, among a wide array of other components. For example, the biometric components 1232 may include components to detect expressions (e.g., hand expressions, facial expressions, vocal expressions, body gestures, or eye tracking), measure biosignals (e.g., blood pressure, heart rate, body temperature, perspiration, or brain waves), identify a person (e.g., voice identification, retinal identification, facial identification, fingerprint identification, or electroencephalogram-based identification), and the like. The motion components 1234 may include acceleration sensor components (e.g., accelerometer), gravitation sensor components, rotation sensor components (e.g., gyroscope), and so forth. The environmental components 1236 may include, for example, illumination sensor components (e.g., photometer), temperature sensor components (e.g., one or more thermometers that detect ambient temperature), humidity sensor components, pressure sensor components (e.g., barometer), acoustic sensor components (e.g., one or more microphones that detect background noise), proximity sensor components (e.g., infrared sensors that detect nearby objects), gas sensors (e.g., gas detection sensors to detection concentrations of hazardous gases for safety or to measure pollutants in the atmosphere), or other components that may provide indications, measurements, or signals corresponding to a surrounding physical environment. The position components 1238 may include location sensor components (e.g., a GPS receiver component), altitude sensor components (e.g., altimeters or barometers that detect air pressure from which altitude may be derived), orientation sensor components (e.g., magnetometers), and the like.

Communication may be implemented using a wide variety of technologies. The I/O components 1242 may include communication components 1240 operable to couple the machine 1200 to a network 1220 or devices 1222 via a coupling 1224 and a coupling 1226, respectively. For example, the communication components 1240 may include a network interface component or another suitable device to interface with the network 1220. In further examples, the communication components 1240 may include wired communication components, wireless communication components, cellular communication components, Near Field Communication (NFC) components, Bluetooth® components (e.g., Bluetooth® Low Energy), Wi-Fi® components, and other communication components to provide communication via other modalities. The devices 1222 may be another machine or any of a wide variety of peripheral devices (e.g., a peripheral device coupled via a USB).

Moreover, the communication components 1240 may detect identifiers or include components operable to detect identifiers. For example, the communication components 1240 may include Radio Frequency Identification (RFID) tag reader components, NFC smart tag detection components, optical reader components (e.g., an optical sensor to detect one-dimensional bar codes such as Universal Product Code (UPC) bar code, multi-dimensional bar codes such as Quick Response (QR) code, Aztec code, Data Matrix, Dataglyph, MaxiCode, PDF417, Ultra Code, UCC RSS-2D bar code, and other optical codes), or acoustic detection components (e.g., microphones to identify tagged audio signals). In addition, a variety of information may be derived via the communication components 1240, such as location via Internet Protocol (IP) geolocation, location via Wi-Fi® signal triangulation, location via detecting an NFC beacon signal that may indicate a particular location, and so forth.

The various memories (i.e., memory 1204, main memory 1212, static memory 1214, and/or memory of the processors 1202) and/or storage unit 1216 may store one or more sets of instructions and data structures (e.g., software) embodying or utilized by any one or more of the methodologies or functions described herein. These instructions (e.g., the instructions 1208), when executed by processors 1202, cause various operations to implement the disclosed embodiments.

As used herein, the terms “machine-storage medium,” “device-storage medium,” “computer-storage medium” mean the same thing and may be used interchangeably in this disclosure. The terms refer to a single or multiple storage devices and/or media (e.g., a centralized or distributed database, and/or associated caches and servers) that store executable instructions and/or data. The terms shall accordingly be taken to include, but not be limited to, solid-state memories, and optical and magnetic media, including memory internal or external to processors. Specific examples of machine-storage media, computer-storage media and/or device-storage media include non-volatile memory, including by way of example semiconductor memory devices, e.g., erasable programmable read-only memory (EPROM), electrically erasable programmable read-only memory (EEPROM), FPGA, and flash memory devices; magnetic disks such as internal hard disks and removable disks; magneto-optical disks; and CD-ROM and DVD-ROM disks. The terms “machine-storage media,” “computer-storage media,” and “device-storage media” specifically exclude carrier waves, modulated data signals, and other such media, at least some of which are covered under the term “signal medium” discussed below.

In various example embodiments, one or more portions of the network 1220 may be an ad hoc network, an intranet, an extranet, a VPN, a LAN, a WLAN, a WAN, a WWAN, a MAN, the Internet, a portion of the Internet, a portion of the PSTN, a plain old telephone service (POTS) network, a cellular telephone network, a wireless network, a Wi-Fi® network, another type of network, or a combination of two or more such networks. For example, the network 1220 or a portion of the network 1220 may include a wireless or cellular network, and the coupling 1224 may be a Code Division Multiple Access (CDMA) connection, a Global System for Mobile communications (GSM) connection, or another type of cellular or wireless coupling. In this example, the coupling 1224 may implement any of a variety of types of data transfer technology, such as Single Carrier Radio Transmission Technology (1×RTT), Evolution-Data Optimized (EVDO) technology, General Packet Radio Service (GPRS) technology, Enhanced Data rates for GSM Evolution (EDGE) technology, third Generation Partnership Project (3GPP) including 3G, fourth generation wireless (4G) networks, Universal Mobile Telecommunications System (UMTS), High Speed Packet Access (HSPA), Worldwide Interoperability for Microwave Access (WiMAX), Long Term Evolution (LTE) standard, others defined by various standard-setting organizations, other long range protocols, or other data transfer technology.

The instructions 1208 may be transmitted or received over the network 1220 using a transmission medium via a network interface device (e.g., a network interface component included in the communication components 1240) and utilizing any one of several well-known transfer protocols (e.g., hypertext transfer protocol (HTTP)). Similarly, the instructions 1208 may be transmitted or received using a transmission medium via the coupling 1226 (e.g., a peer-to-peer coupling) to the devices 1222. The terms “transmission medium” and “signal medium” mean the same thing and may be used interchangeably in this disclosure. The terms “transmission medium” and “signal medium” shall be taken to include any intangible medium that can store, encoding, or carrying the instructions 1208 for execution by the machine 1200, and includes digital or analog communications signals or other intangible media to facilitate communication of such software. Hence, the terms “transmission medium” and “signal medium” shall be taken to include any form of modulated data signal, carrier wave, and so forth. The term “modulated data signal” means a signal that has one or more of its characteristics set or changed in such a matter as to encode information in the signal.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

By using genuine models of anatomy more accurate surgical plans may be developed than through statistical modeling.

Terms used herein should be accorded their ordinary meaning in the relevant arts, or the meaning indicated by their use in context, but if an express definition is provided, that meaning controls.

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to one or multiple ones. Additionally, the words “herein,” “above,” “below” and words of similar import, when used in this application, refer to this application as a whole and not to any portions of this application. When the claims use the word “or” in reference to a list of two or more items, that word covers all the following interpretations of the word: any of the items in the list, all the items in the list and any combination of the items in the list, unless expressly limited to one or the other. Any terms not expressly defined herein have their conventional meaning as commonly understood by those having skill in the relevant art(s).

What is claimed is:

1. An endoscope camera controller, comprising:
a processor and memory coupled to the processor, the memory comprising instructions, which when executed by the processor cause the endoscope camera controller to:
receive an indication of a pulse start control signal and one or more laser pulse characteristics, the pulse start control signal to define an initiation of lasing by a lasing console and the one or more laser pulse characteristics to define the lasing;
determine an integration timing period of a camera sensor of an endoscope based on the pulse start control signal and the one or more laser pulse characteristics; and
generate an integration control signal, the integration control signal to cause the camera sensor to accumulate charge during integration timing period.

2. The endoscope camera controller of claim 1, the instructions when executed by the processor further cause the endoscope camera controller to send the integration control signal to shutter gating circuitry of the endoscope, the shutter gating circuitry to control a shutter of the endoscope camera.

3. The endoscope camera controller of claim 1, wherein the one or more laser pulse characteristics define a frequency of pulses to be generated by the lasing console.

4. The endoscope camera controller of claim 3, wherein the one or more laser pulse characteristics define a pulse width of the pulses.

5. The endoscope camera controller of claim 4, the instructions when executed by the processor further cause the endoscope camera controller to:
determine a maximum duration of integration of the camera sensor based on a frame rate of the camera; and
determine the integration timing period as the difference of the maximum duration of integration of the camera and the pulse width.

6. The endoscope camera controller of claim 1, wherein the camera is a global shutter style camera or a rolling shutter style camera.

7. The endoscope camera controller of claim 1, wherein the lasing console comprises a Thulium fiber laser source or a Holmium laser source.

8. A laser console, comprising:
a processor and memory coupled to the processor, the memory comprising instructions, which when executed by the processor cause the laser console to:
receive an indication of settings of an endoscope camera;
determine laser pulse characteristics and a pulse start time for a laser source of the laser console based on the settings; and
cause the laser source to generate pulses of light based on the laser pulse characteristics and the pulse start time.

9. The laser console of claim 8, wherein the settings comprise an indication of a frame rate and a vertical blanking period of the endoscope camera.

10. The laser console of claim 9, the instructions when executed by the processor further cause the laser console to determine the pulse start time based on the vertical blanking period.

11. The laser console claim 8, the instructions when executed by the processor further cause the laser console to determine the laser pulse characteristics comprises determining a frequency of the laser source based on a factor of the frame rate.

12. The laser console of claim 8, wherein the laser source is a Thulium fiber laser source.

13. The laser console of claim 8, wherein the laser source is a Holmium laser source.

14. The laser console of claim 8, wherein the endoscope camera is a global shutter style camera or a rolling shutter style camera.

15. An endoscope console, comprising:
a processor and memory coupled to the processor, the memory comprising instructions, which when executed by the processor cause the endoscope console to:
receive an indication of settings of an endoscope camera;
receive an indication of a pulse start time and one or more laser pulse characteristics, the pulse start control signal to define an initiation of lasing by the lasing console and the one or more laser pulse characteristics to define the lasing;
receive a plurality of image frames from a camera of an endoscope coupled to the endoscope console;
identify at least a portion of a one of the plurality of image frames where lasing by the lasing console overlaps with integration by the camera based on the one or more laser pulse characteristics, the pulse start time, and the settings;
identify at least a portion of another one of the plurality of image frames where lasing by the lasing console does not overlap with integration by the camera based on the one or more laser pulse characteristics, the pulse start time, and the settings; and
replace at least the portion of the one of the plurality of image frames with at least the portion of the other one of the plurality of image frames, wherein the other one of the plurality of image frames is prior in time to the one of the plurality of image frames.

16. The endoscope console of claim 15, wherein the settings comprise an indication of a frame rate and a vertical blanking period of the endoscope camera.

17. The endoscope console of claim 15, wherein the one or more laser pulse characteristics define a frequency of pulses to be generated by the lasing console.

18. The endoscope console of claim 17, wherein the one or more laser pulse characteristics define a pulse width of the pulses.

19. The endoscope console of claim 15, wherein the camera is a global shutter style camera or a rolling shutter style camera.

20. The endoscope console of claim 15, wherein the lasing console comprises a Thulium fiber laser source or a Holmium laser source.
